(19) United States
(12) Patent Application Publication
(10) Pub. No.: US 2002/0048374 A1
(43) Pub. Date: Apr. 25, 2002

(54) Method and apparatus for measuring the performance of an implantable middle ear hearing aid, and the respones of a patient wearing such a hearing aid

(76) Inventors: Sigfrid Soli, Sierra Madre, CA (US); Douglas Alan Miller, Lafayette, CO (US); Stanley A. Woodard, Boulder, CO (US); Scott Allen Miller III, Boulder, CO (US)

Correspondence Address: MARSH FISCHMANN & BREYFOGLE LLP, Suite 411, 3151 South Vaughn Way, Aurora, CO, 80014, US

(21) Appl. No.: 09/872,079

(22) Filed: Jun. 1, 2001

Related U.S. Application Data
(60) Provisional application No. 60/209,006, filed on Jun. 1, 2000.

(51) Int. Cl. H04R 29/00; A61B 5/00

(52) U.S. Cl. 381/60; 600/559

(57) ABSTRACT

A reference transmitter (602) and reference receiver (604) are provided for testing the performance of a semi-implantable hearing aid. In a calibration configuration, an audiometer (606) is used to provide a reference signal via a headphone jack output (608) to the reference transmitter (602). The reference transmitter (602) provides an RF transmit coil output via lead (610) and coil (612) to the reference receiver (604). The reference receiver (604) provides an output signal that is correlated to a microphone signal to a hearing aid analyzer (616). The transmitter (602) and receiver (604) can be separately used to analyze the internal and external portions of a semi-implantable hearing aid using conventional audiometers and hearing aid analyzers.

### RELATED APPLICATION INFORMATION

This application claims priority from U.S. Provisional Patent Application Ser. No. 60/209,006 filed on Jun. 1, 2000, which is incorporated herein by reference.

### FIELD OF THE INVENTION

The present invention relates in general to testing of hearing aids and, in particular, to testing the performance of middle ear hearing aids, including an implantable portion, such as a semi-implantable electromechanical transducer hearing aid, especially in situ.

### BACKGROUND OF THE INVENTION

The purpose of a hearing aid is to compensate for a patient's loss of hearing function and, especially, to enhance the patient's intelligibility scores, i.e., their ability to understand speech. This is done via detecting the ambient acoustic signals, processing them according to a prescription, and delivering the processed signal to the patient in a manner that the patient then perceives as sound. Hearing aids differ in the manner in which the signal is processed and the processed signal is delivered to the patient.

The processing step, known as Speech Signal Processing (SSP), may include a number of steps, such as amplification, frequency shaping, compression, et cetera. The steps in the SSP are determined by the design of the hearing aid, while the particular internal values (IV) used in the steps are generated from prescriptive parameters (PP) determined by the audiologist. Thus, the number of frequency bands used by a hearing aid are determined by the design, while the desired amount of attenuation of each frequency band is given as a prescriptive parameter, and the actual numbers used in the hearing aid to set these frequency attenuations are the internal values. It will be appreciated that some hearing aids provide the ability to select which SSP steps are performed, in which case the configuration is part of the IV, as well as the PP.

Once the ambient acoustic signal is processed by the SSP, the altered signal stimulates the patient through a transducer. This may be done acoustically, mechanically, or via nerve stimulation. If the patient's own ear canal is used for acoustic stimulation, there is no need for implanting a device within the patient. On the other hand, if electrical or mechanical stimulation is used, some mechanism is needed for optimizing the quality of the signal from the transducer, which mechanism therefore frequently is needed to be in direct contact with one or more of the structures responsible for the perception of hearing.

The most common type of hearing aid is the external hearing aid, using an acoustic transducer. Common varieties of external hearing aids may be worn behind the ear (BTE), in the ear canal (ITC), or completely in the canal (CIC). In addition to using acoustic transducers, these all have in common that none of the apparatus is implanted within the body, nor is in contact with the bloodstream.

The type of implanted hearing aid with which the public is currently most familiar is the cochlear implant. This uses one or more electrodes to directly stimulate the nerves of the cochlea, causing the sensation of sound. Each electrode corresponds roughly to a particular frequency and the degree of stimulation of an area corresponds roughly to the sound amplitude, but these correspondences are, in fact, much more complex. Additionally, these correspondences are confused by particulars of the physiology and psychoacoustics of a given patient, which are non-linear. Subsequently, cochlear implants require an additional processing step after the desired signal is generated by the SSP in order to map the acoustic signal into a given pattern of electrodes. There is a learning period after the fitting of the implant, in which the mapping is made more perfect in the short term by the adaptation of the hearing aid to the patient and in the long term by the patient's brain to the hearing aid.

Yet another type of implantable hearing aid uses brainstem stimulation to perform a similar service for the patient as a cochlear implant. In this case, however, the correspondences between the electrical stimulus and various acoustical parameters are very involved, highly nonlinear and are unknown for a given patient; in fact, this mapping task is one of the most difficult for brainstem stimulation and has not yet been satisfactorily addressed. As a result, the quality of perceived sound from a brainstem stimulation implant is presently very crude.

Another general type of hearing aid is middle ear stimulation using mechanical vibration. In this hearing aid, one or more bones of the middle ear (the ossicles) are made to mechanically vibrate, causing the vibration to stimulate the cochlea through its natural input, the so-called oval window. An example of such a hearing aid is the MET™ hearing aid of Otologics, LLC, developed by Fredrickson et al in which a small electromechanical transducer is used to vibrate the incus (the 2^{nd} of the 3 bones forming the ossicles), and thence produce the perception of sound.

A hearing aid which uses an implanted transducer to stimulate some portion of the hearing process may be of either one of two classifications: fully implantable, in which the hearing aid is self-contained within the patient, or semi-implantable, in which some of the components, typically the microphone, power supply, and speech signal processing, are external to the patient, while the transducer and key support functions are implanted. The two pieces of a semi-implantable hearing aid communicate via some type of communications channel, typically wireless in nature. The external portion of a semi-implantable hearing aid are normally worn as a BTE.

It will be appreciated that since with a middle ear transducer, the cochlea is being stimulated via its natural input, and since the ossicular chain and tympanic membrane are largely linear in response characteristics, the mapping problem for a middle ear hearing aid from desired output to stimulation is greatly simplified relative to, as well as being very different from the mapping process for either a cochlear implant or a brainstem implant. At the same time, the output of a middle ear transducer is considerably different from the output of an external hearing aid in that the output is not conveniently accessible for measurement, nor is it amenable to measurement with standard audiological laboratory instruments or practices. Therefore, a new system of testing instruments, processes and standards are required for middle ear hearing aids. In order to minimize the learning curve for the audiologist, such instruments, processes and standards should be largely analogous to their present practice using external hearing aids.

In adapting a given external hearing aid to a given patient, the various PP must be chosen to provide the most benefit to the patient, and are typically determined by a process known as fitting. This fitting process comprises determining various measures of the patient's unaided hearing perception, generating the desired compensation as PP via a fitting algorithm, or simply algorithm. Continuing the fitting process, the PP are then converted to IV for the hearing aid, the hearing aid is programmed with these IV, and then verifying that these IV demonstrably correspond to the desired PP. Once this is completed, the hearing aid is placed on the patient and various measures of the patient's aided hearing perception are determined to find out if the fitting process has been successful. If the patient's aided hearing perception is within acceptable limits the fitting is completed. Otherwise, the audiologist may elect to alter either the PP or the IV from the prescribed values slightly in order to attempt to improve the results for the patient.

In the case of an external hearing aid, the patient's unaided hearing perception may be measured by subjecting the patient to various sound test protocols well known to those skilled in the art. These test protocols consist of sounds presented to the patient via speakers or headphones in a soundproof booth. The sounds may consist of tones, composite tones, multiple tones, speech, or the like, and they may be presented to one or both of the ears. For example, a common measurement of a patient's hearing perception is to subject the patient to a sequence of pure tones at specific “audiometric”frequencies. A device known as an audiometer is used to generate this sequence of tones as electrical signals which are thence conducted by a cable to the speakers or headphones.

These tones are presented to the subject at various amplitudes according to specific protocols used in the industry, the purpose of which is to determine the quietest sound the patient can hear, called the Hearing Threshold Level (HTL). These tones are presented to the ear under test (EUT), while the opposite ear is typically “muffled and masked” meaning enclosed in a headphone which both seals out external sound and simultaneously exposes that ear to white noise which confounds or “masks” the perception of any sound which leaks through the headphone. With the opposite ear thus muffled and masked, the audiologist can be assured that the response of the patient is due to the EUT and not the response of the opposite ear.

By elevating the acoustic output of the hearing aid due to a normal conversation to the patient's perception of a normal conversation, one might expect to compensate for hearing loss. One way of estimating this might be by measuring the difference between a normal HTL and the patient's HTL, and setting the gain of the hearing aid to that amount. Such a hearing aid is called linear.

Unfortunately, the loudest sound the patient can comfortably tolerate, called the UnComfortableness Level (UCL), does not go up by the same amount as the change in HTL. In fact, it typically stays at the same level, or even goes down. As a result, providing the same gain for all input levels would cause uncomfortable or even painful levels of stimulation for loud input sounds. Thus, the audiologist typically measures the patient's UCL as well as the HTL.

An audiologist may also attempt to measure the relationship between various amplitudes of sounds and the relative size of the perceived amplitudes. This “loudness growth function” may be measured in various ways, but one way is the presentation of two tones. One of these tones would be a reference tone, for example, a 1 kHz tone at 70 dB SPL. The second tone would typically be at an audiometric frequency. Each tone is presented alternately to the patient, with the amplitude of the second tone adjusted until the patient perceives both tones as having the same amplitude. In like manner, the loudness growth of each appropriate audiometric frequency is determined.

Once the appropriate unaided audiometric measures are performed, a fitting algorithm is used to convert this data into the most appropriate mapping between the patient's hearing and normal hearing. This process is not as simple as it sounds. In our example, fitting the obvious naive technique is to map the patient's HTL to the normal HTL and the patient's UCL onto the normal UCL for all audiometric frequencies, using frequency shaping and compression as needed. Unfortunately, this technique is usually unsatisfactory, as it typically results in the ratios of energy in various frequency bands being disturbed relative to each other. Since speech intelligibility depends critically on the relative ratios of certain frequency bands being maintained, the result of such a naive fitting is to destroy the patient's ability to distinguish between various phonemes.

In order to prevent or at least mitigate this loss of intelligibility, various philosophies exist. These philosophies are reduced to a fitting algorithm, or simply algorithm, which is used to perform the actual calculation. For example, not modifying the patient's hearing response at all results in loss of intelligibility due to, perhaps, normal conversations being below the patient's threshold of hearing, but a naive fitting is unsatisfactory due, perhaps, to alteration of the relative ratios of frequency bands. A simple algorithm might be to correct, instead of to normal hearing, to a weighted combination between the patient's unaided hearing and normal hearing, while attempting to map a normal conversation to the patient's comfortable level of hearing. Various schools of thought exist as to the best fitting algorithms, and the range of their applicability. The results of the algorithm is a set of mapping parameters describing how to map the acoustic input into the patient's perception as prescriptive parameters.

Once this is done, the prescriptive parameters must be converted into parameters suitable for use inside of the hearing aid. Depending on the technology used in the speech signal processing, this results in numbers, here called internal values, which are then programmed into the hearing aid. This function is often included in the function of the fitting software purchased by the audiologist. The programming activity itself is done from a universal hearing aid programmer, such as the HiPro® from Madsen Electronics of Denmark.

Before the external hearing aid is programmed with the desired internal values, the audiologist will often verify the proper functioning of the hearing aid according to the manufacturer's instructions. This may involve putting a particular program into the hearing aid, and measuring its performance on a hearing aid analyzer. This device tests the hearing aid in a sound-reducing chamber with a speaker. The acoustic hearing aid output is conducted to a device used to simulate the acoustic properties of the ear canal, for example a 2 cc coupler, and thence to a microphone. The hearing aid is then subjected to a series of tests, such as those specified in ANSI S3.22-1996, whose purpose to verify that it conforms to the performance of a properly functioning aid within a set tolerance.

After the operation of the hearing aid is confirmed, the appropriate internal values are programmed into the hearing aid, and the device is once again placed in the hearing aid analyzer. The expected performance of the desired program is then confirmed by comparing the actual response of the programmed device with the desired performance. This confirms that the patient will be receiving at least approximately the desired amount of hearing compensation by the aid, will not be subjected to an excessive amount of acoustic energy, and that the performance of the aid will be suitable to warrant further tests with the patient. If the hearing aid produces the desired response, the aid will be placed on the patient for testing.

If, as occasionally happens, the hearing aid has been found to be in good working condition but the actual response of the device as determined by the hearing aid analyzer is different from the desired response by a significant amount, the audiologist may elect to adjust the programmed internal values, or somewhat equivalently, the prescriptive parameters. This capability is frequently provided by the hearing aid manufacturer, and may be part of the fitting software. It is necessary to perform this test and subsequent adjustment because the speech signal processing of hearing aids is simply an approximation to the performance of an ideal speech signal processing. For example, the frequency shaping performed by a hearing aid does not typically have perfect independence between each frequency band, but demonstrates interactions. These interactions are such that increasing the amplitude of one frequency band may, for instance, increase the amplitude of frequencies that are adjacent to that band. To some extent, this can be compensated for in software, but in fact, there are some frequency shaping curves that are not possible for a given hearing aid, but can only be approximated.

Once the aid is placed on the patient, similar acoustic tests as were performed on the unaided ear are performed on the patient using the aid. This allows the audiologist to confirm that the aid is compensating the deficient hearing appropriately. If the patient and audiologist agree that the performance is satisfactory, the patient will be sent home with the device. If, on the other hand, the patient feels the aid performance is uncomfortable, the audiologist may elect to send the patient home with the aid as-is anyway, as an adaptation by the patient to the new hearing performance may be required, or the audiologist may choose to adjust the programmed internal values or nearly equivalently the prescribed parameters. Through this process, an acceptable level of performance is arrived at, at which point the patient may be released with the aid.

Throughout this process of fitting an acoustic hearing aid to a patient, in order to be able to compare the patient's measurements with normal measurements, and to confirm the proper operation of the hearing aid, the acoustic equipment, including the audiometer, headphones, microphone, etc. needs to be calibrated. Unfortunately, the requisite system of equipment for measuring and maintaining calibration of the measurements does not exist for middle ear implants. Specifically, the implanted hearing aid cannot be tested for satisfactory performance when implanted in the patient and receiving information from the communications channel. Moreover, the implantation process itself or the progression of pathology may alter the performance of the implant, further complicating the establishment and maintenance of calibration. While it is possible to perform the implantation, measure the patient's perception with speakers or headphones, and adjust the parameters of the device until it is working successfully, with the current state-of-the-art it is not possible to 1) verify that both the internal and external components of the aid are operating properly 2) measure the performance of the aid once implanted and 3) compare the results with normal hearing patients. Hence, it is not possible to 4) successfully calculate prescriptive parameters based on a fitting algorithm, nor 5) verify that the aid conforms to the performance required by the fitting algorithm independently of the patient.

This invention discloses a method which allows steps 1) and 2) above to be performed (and thereby steps 3, 4 and 5), in part by providing suitable instrumentation for the direct stimulation of the implant portion of the aid via the communications channel, and for the measurement of the communications channel stimulation provided by the external portion of the aid. This puts the fitting process for middle ear implants onto a scientific basis, and additionally accrues several other advantages. These include greater exclusion of noise from the system, the ability to compare data from different sites easily, and greater comfort.

### SUMMARY OF THE INVENTION

The present invention is directed to a method and apparatus for measuring the performance of the internal and external portion of a semi-implantable hearing aid such as an electromechanical transducer hearing aid. The invention provides calibrated measurements that are repeatable and verifiable across sites. In addition, the invention allows for evaluation of the perception of the patient through the implant while bypassing the other ear, the tympanic membrane and the malleus thereby allowing measurement of the device stimulation path only. The invention enables measurement of semi-implantable device performance utilizing components of proven testing equipment and standards developed for external, acoustical hearing aids.

According to one aspect of the present invention, an apparatus (hereinafter termed a reference transmitter) is provided for use in evaluating the perception of the patient through the implant. The implanted hearing aid element is adapted for directly stimulating a middle ear element of a patient, for example, the incus, in response to a communications channel such as an RF signal transmitted transcutaneously to the implanted hearing aid element. The reference transmitter includes an input port for receiving an input signal reflecting a test acoustical output of an audiometer, a converter system for converting the input signal into an output signal representing a test communications channel signal and an output port for outputting the communications channel signal adapted for placement over the implanted hearing aid element on a head of a patient. Upon transmission of the communications channel signal to the implanted hearing aid element, the perception of the patient through the implant can be analyzed, and in this manner, conventional audiometers with the calibrated apparatus can be employed.

A corresponding operating process of the present invention is provided for use in evaluating the perception of the patient through the implant. The method includes the steps of placing a test signal output device over the implanted hearing aid element on the head of a patient, operating an audiometer, reference transmitter and a reference signal output device to transcutaneously transmit the test communication signal to the implanted hearing aid element, soliciting feedback from the patient regarding the perception of the transmitted test communication signal and adjusting the hearing aid based on the feedback from the patient.

According to another aspect of the present invention, an apparatus (hereinafter termed a reference receiver) is provided for use in testing an external portion of a semi-implantable hearing aid. The external portion is adapted for transcutaneously transmitting communication signals (such as electromagnetic signals) to an implanted portion of the hearing aid. The reference receiver includes an input port for receiving an input communication signal from the exterior portion of the hearing aid, a signal processor for processing the input communication signal to generate an output signal and an output port for providing the output signal to a commercial hearing aid analyzer or the like.

A corresponding operating process of the present invention is provided for use in testing an external portion of a semi-implantable hearing aid. The input communication signal is based on a test acoustical signal provided by a hearing aid analyzer and reflects the signal that would be provided by the exterior portion when mounted on a head of patient in a similar test acoustic field. The output signal amplitude preferably corresponds to the microphone signal level of an external acoustical hearing aid testing system under the equivalent acoustic amplitude conditions. The hearing aid analyzer uses the output signal to evaluate a performance of the exterior portion of the hearing aid. By virtue of the invention, a hearing aid analyzer that has been developed for use in testing external, acoustical hearing aids can be utilized to test the external portion of the semi-implantable hearing aid.

Yet another associated method involves receiving an input signal reflecting a test acoustical output of an audiometer, converting the signal into a test communications channel signal with a reference receiver, transmitting the communications channel signal to a reference receiver via a communications channel, and providing an output of the reference receiver adapted to the input of a standard microphone input of a commercial hearing aid analyzer or the like. In this manner, the performance of the reference transmitter coupled to the reference receiver may be evaluated, for instance for purposes of calibration and determining that both are in good working order.

### BRIEF DESCRIPTION OF THE DRAWINGS

For a more complete understanding of the present invention and further advantages thereof, reference is now made to the following detailed description taken in conjunction with the drawings, in which:

FIG. 1 illustrates a semi-implantable hearing aid mounted in the head of a patient;

FIG. 2 illustrates a reference receiver in accordance with the present invention for measuring the performance of an external portion of a semi-implantable hearing aid;

FIG. 3 illustrates the reference receiver of FIG. 2 set up for measuring the performance of an exterior portion of a semi-implantable hearing aid;

FIG. 4 illustrates a reference transmitter system in accordance with the present invention;

FIG. 5 illustrates a reference transmitter and reference receiver in accordance with the present invention set up for a calibration process.

### DETAILED DESCRIPTION

In the following description, the invention is set forth in the context of a reference transmitter and reference receiver used for testing the performance of a semi-implantable hearing aid. Although specific embodiments and implementations are described, it will be appreciated that certain aspects are more broadly applicable in a variety of hearing aid testing environments. Accordingly, the following description should be understood as exemplifying but not limiting the scope of the invention.

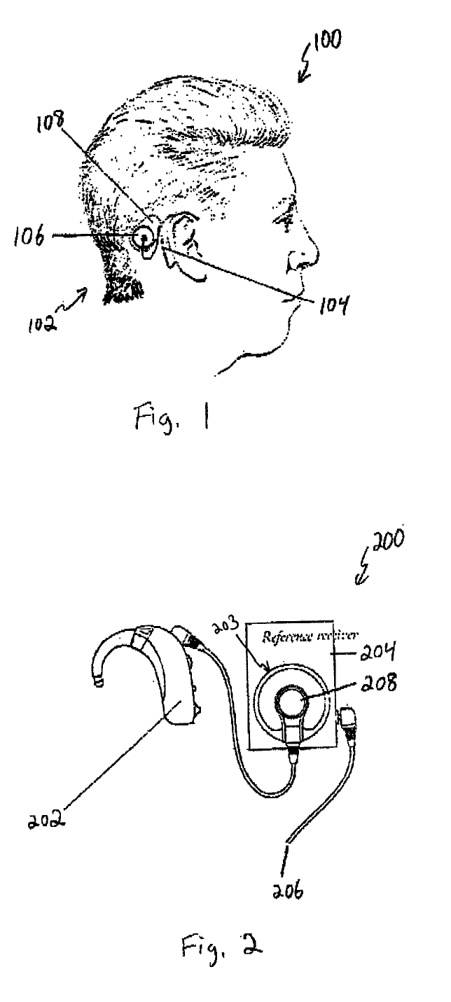

Referring to FIG. 1, a semi-implantable hearing aid 100 is illustrated. The hearing aid generally includes an external portion 102 and an interior portion 108. The exterior portion includes an acoustical signal receiver-transducer 104 adapted to be worn on the outer ear and a radio transmitter element 106 that is mounted on the patient's head behind the ear overlying the internal portion 108. The external portion 102 receives acoustic signals, generates an RF signal representative of the received acoustical signals and transcutaneously transmits the RF signals via a radio transmitter element 106 to the internal portion 108. The internal portion 108 directly stimulates the middle ear. For example, in the case of an electromechanical transducer hearing aid, the internal portion 108 includes a receiver for detecting the RF signal and an electromechanical transducer for driving a mechanical element in response to the received RF signal. The mechanical element, in turn, drives the incus of the ossicular chain which is perceived by the patient as sound. It will be appreciated that this mechanical driving of the ossicular chain supplements driving of the ossicular chain by the tympanic membrane as part of the patient's natural hearing process. Elements of such a semi-implantable hearing aid are described in U.S. Pat. No. 5,702,342, which is incorporated herein by reference.

It will be appreciated that the overall performance of the hearing aid 100 is dependent on both the operation of the external portion 102 and the internal portion 108. That is, in understanding and enhancing the operation of the hearing aid 100, it is useful to measure the performance of the external portion of 102 in generating an RF signal representative of a received acoustical signal and to measure the performance of the internal portion 108 in generating a mechanical signal representative of the received RF signal. As set forth in detail below, the present invention provides structure and associated methodology for measuring the performance of the external portion 102 and internal portion 108.

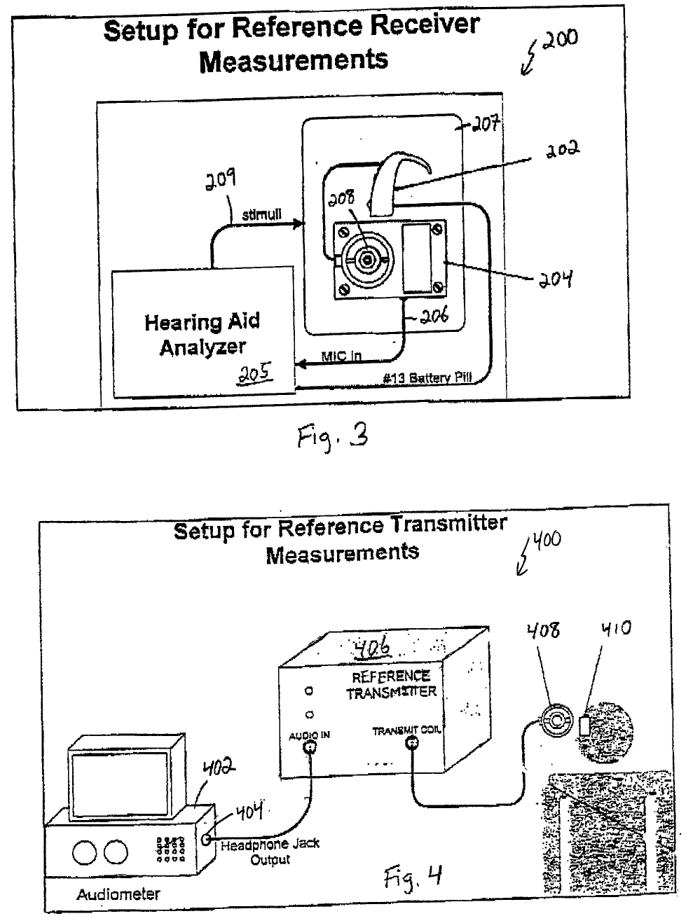

FIGS. 2 and 3 illustrate a reference receiver system 200 for use in measuring the performance of an external portion 202 of a hearing aid under analysis. In particular, the reference receiver system 200 includes a reference receiver unit 204 and an output lead 206 for connecting the reference receiver 204 to a hearing aid analyzer 205. For example, the analyzer 205 may be a conventional hearing aid analyzer marketed by Frye Electronics. The analyzer 205 allows for measurement and calibration of the frequency response, gain, output and compression of the external portion 202.

The illustrated reference receiver system 200 receives an output signal from the external portion 202 and provides an electrical output signal via the output lead 206 to the microphone input of a conventional external, acoustic hearing aid analyzer system. Accordingly, the reference receiver unit 204 includes components for receiving the RF signal in a manner substantially identical to the receiving process of an average implant, and converting it into an electrical output analogous to the mechanical output of an electromechanical transducer as loaded by a model ossicular chain. The electrical components of this electrical analog are selected by a design process in which the electrical impedance of a loaded electromechanical transducer is measured with an impedance bridge, and the equivalent elements are determined by fitting the data to the electrical model. These equivalent elements are then physically built into the reference receiver 204 following the circuitry of the electrical model. This design process is performed only once, and results in the same equivalent elements in all constructed reference receivers as long as the same electromechanical transducer is used in all implants. The receiver unit 204 also includes an input port, generally indicated at 203, such as a recess in, or designated surface of, the external surface of the receiver unit 204 for engaging the external unit 202 such that a radio transmitter element 208 of the external unit 202 is engaged in aligned registration with the transducer of the reference receiver unit 204, and spaced at a distance equivalent to the average spacing between the RF receiving area of an implant 108 (FIG. 1) and radio transmitter element 106. The average is performed over the population of patients expected. An alternative embodiment allows the spacing between the transducer of the reference receiver unit 204 and the radio transmitter element 208 to be adjustable, and can be set to the expected or actual distance found in a given patient.

The present invention advantageously allows for utilization of a conventional analyzer 205 adapted for external, acoustical hearing aid analysis for testing the external portion of a semi-implantable hearing aid. Thus, the output lead 206 is coupled directly to the microphone input of such a hearing aid analyzer. In order for the analyzer 205 to provide a meaningful analysis in the case of an external portion of a semi-implantable hearing aid device as illustrated, the circuitry of the reference receiver unit 204 processes the electrical signal from the transducer such that the characteristics of the resulting output signal are substantially mapped to physiologically corresponding characteristics of conventional microphone signals. Over the course of many samples over a significant period of time, the designers of testing units for external, acoustical hearing aids have theoretically and empirically derived relationships relating microphone signals to normal patient sound perception. Similarly, through theoretical and empirical investigation, it is possible to design the reference receiver unit 204 such that the signals from the transducer are translated into output signals that correspond to microphone signals and, in turn, to patient sound perception. In this manner, the reference receiver unit 204 allows the external portion of a semi-implantable hearing aid to be tested in a manner analogous to the testing of external, acoustical hearing aids using existing hearing aid analyzers. Moreover, the reference receiver provides calibrated measurements that are repeatable and verifiable across sites.

Thus, an external portion 202 of a hearing aid under analysis can be tested by: placing the external portion 202 into a test chamber 207 of a hearing aid analyzer 205; placing the transmitter element 208 the desired distance (as described above) from the input surface of the reference receiver unit 204, connecting the output lead 206 of the reference receiver 204 to the microphone jack of the analyzer 205; connecting an input lead 209 between the analyzer 205 and the chamber 207 to conduct a test electrical signal to the chamber 207 where the test electrical signal is converted into a test acoustic signal, operating the analyzer 205 to provide the test acoustical signal to the external hearing aid portion 202 in the chamber 207; receiving a resulting signal from the external hearing aid portion using the reference receiver unit 204 to provide an output signal corresponding to a conventional microphone signal; and operating the analyzer 205 to analyze the output signal and provide information regarding the performance of the external hearing aid portion 202 under analysis.

As noted above, in order to properly program the hearing aid, it is also necessary to measure the patient's perceived response to the performance of the implanted hearing aid portion. It has been recognized that measurement of the patient's perceived response to the performance of the implanted hearing aid portion can be enhanced by providing a test signal to the implanted portion without utilizing hearing aid external portions that may vary from unit to unit, may have limited acoustic performance, and also bypassing the outer ear, the tympanic membrane and the malleus. This is accomplished in accordance with the present invention by using a reference transmitter system 400 as shown in FIG. 4. The illustrated system 400 includes an audiometer 402, with a headphone output module generally indicated at 404, a reference transmitter unit 406 and a radio transmitter element 408 such as a transmitter coil.

The illustrated system 400 advantageously utilizes a conventional audiometer 402 designed for testing the patient's perceived response to the performance of the implanted hearing aid portion. In this regard, the audiometer 402 generates signals representative of a test acoustical pattern. That is, the audiometer 402 provides signals that, when played over headphones (in conventional usage), have known acoustical characteristics in terms of frequency response, amplitude and the like. The illustrated reference transmitter unit 406 receives these headphone signals and processes the headphone signals to drive the radio transducer element 408 so as to provide an RF signal to the implanted hearing aid element 410 that corresponds physiologically to the acoustical signals that are output by headphones in conventional devices. It will thus be appreciated that the illustrated reference transmitter system 400 allows clinicians or other users to employ conventional audiometers 402 for analyzing the performance of an implanted hearing aid element 410. Moreover, the use of a reference transmitter 406 having standardized characteristics allows for calibrated measurements that are repeatable and verifiable across sites. Stimulation of the implanted element 410 via the reference transmitter unit 406 and transmitter element 408 allows for testing of the implanted element 410 free from any variation associated with external hearing aid portions and by activating only the middle ear stimulation path, bypassing the outer ear, the tympanic membrane and the malleus. Accordingly, the performance of the implanted element 410 can be more directly and accurately measured. Based on the measured performance characteristics, the settings of an associated external hearing aid element can be programmed so that the overall performance characteristics of the hearing aid device are mapped to the patient's auditory dynamic range and hearing enhancement needs.

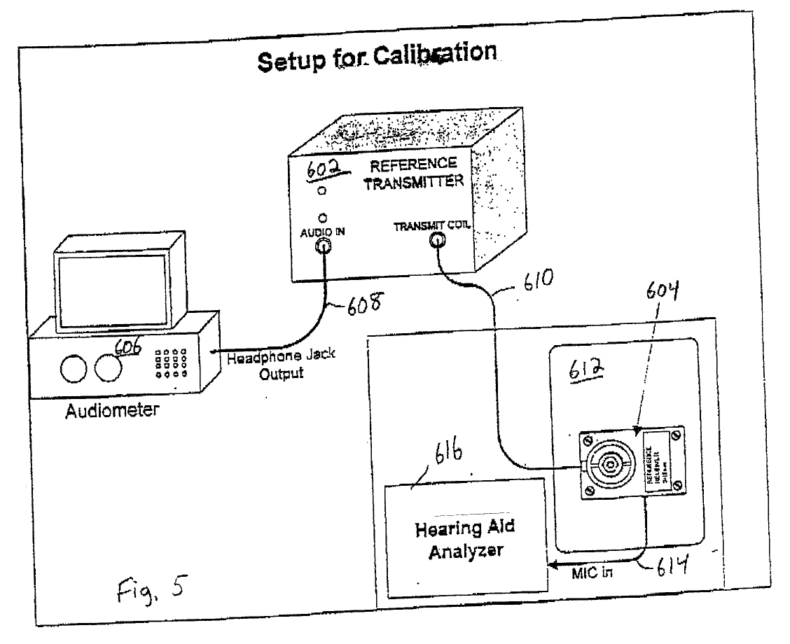

FIG. 5 illustrates a setup of a reference transmitter 602 and reference receiver 604 for calibration. In particular, an audiometer 606 is used to provide a reference signal as discussed above via a headphone jack output 608. The reference transmitter 602 receives the headphone jack signal and provides an RF transmit coil output via lead 610. The RF transmitter coil 612 is engaged with the reference receiver 604 as discussed above. The reference receiver receives the resulting RF signal and provides an output signal that is correlated to a microphone output signal via lead 614. The output signal is provided to a conventional hearing aid analyzer 616 which analyzes the signal to provide performance measurements. Before the reference transmitter 602 is used to measure a patient's thresholds, it is calibrated by connecting it with the reference receiver 604 as shown, with the output measured by a standard hearing aid analyzer 616. In this manner, when the patient's thresholds and uncomfortable loudness levels are known, the output levels of the processor of the external hearing aid portion can be set, verified and documented with the reference receiver 604 before the external hearing aid portion is given to the patient. This process ensures both safety and appropriate amplification.

While various embodiments of the present invention have been described in detail, it is apparent that further modifications and adaptations of the invention will occur to those skilled in the art. However, it is to be expressly understood that such modifications and adaptations are within the spirit and scope of the present invention.

What is claimed is:

1. An apparatus for use in evaluating a patient's response with an implanted element of a hearing aid, said implanted hearing aid element being adapted for directly stimulating a middle ear element of said patient in response to a communication signal transmitted transcutaneously to said implanted hearing aid element, said apparatus comprising:
an input port for receiving an input signal reflecting a reference acoustical output of an audiometer;
a converter system for converting said input signal into an output signal representing a test communication signal; and
an output port for outputting an output signal representative of said test communication signal;
wherein, upon transcutaneous transmission of said test communication signal to said implanted hearing aid element, a performance relative to said patient's response can be analyzed.

2. An apparatus as set forth in claim 1, wherein said input port is interconnected to a headphone output module of said audiometer and said input signal is representative of a test acoustical pattern.

3. An apparatus as set forth in claim 1 where said converter comprises a reference transmitter for driving an RF transmitter to provide said test communication signal based on said input signal reflecting said reference acoustical output of said audiometer.

4. An apparatus as set forth in claim 1, wherein said output port is interconnected to a radio transducer element for generating said output signal as an RF signal.

5. A method for use in testing an implanted element of a hearing aid, said implanted hearing aid element being adapted for directly stimulating a middle ear element of a patient in response to a communication signal transmitted transcutaneously to said implanted hearing aid element, said method comprising the steps of:
receiving an input signal reflecting a test acoustical output of an audiometer;
converting said input signal into a test communication signal;
transmitting the test communication signal via a transmitter adapted for placement over the implanted hearing aid element on a head of a patient;
wherein a performance of the implanted hearing aid element can be evaluated based on said transmitted test communication signal free from acoustical stimulation of the tympanic membrane for testing purposes.

6. A method as set forth in claim 5, wherein said step of converting comprises generating an output signal based on said test acoustical output, for driving said transmitter and driving said transmitter to provide said test communication signal as an RF signal.

7. A method as set forth in claim 5, wherein said step of transmitting comprises positioning said transmitter on the head of said patient proximate to said implanted element and operating said transmitter to transmit said communication signal to said implanted element.

8. A method as set forth in claim 5, further comprising the steps of receiving feedback from said patient regarding a perception of said communication signal and using said feedback to determine a desired performance-related parameter for said hearing aid.

9. A method as set forth in claim 8, wherein said step of using said feedback comprises determining internal values for operation of said hearing aid.

10. A method for use in testing an implanted element of a hearing aid, said implanted hearing aid element being adapted for directly stimulating a middle ear element of a patient in response to a communication signal transmitted transcutaneously to said implanted hearing aid element, said method comprising the steps of:
providing an audiometer, a reference transmitter and a reference signal output, where the audiometer, reference transmitter and reference signal output are operatively interconnected so that the audiometer provides an input signal to the reference transmitter reflecting a test acoustical signal of the audiometer, the reference transmitter converts the input signal into a test communication signal reflective of the reference acoustical signal, and the reference signal output transmits the test communication signal;
placing the reference signal output over the implanted hearing aid element on the head of said patient;
operating the audiometer, reference transmitter and reference signal output to transcutaneously transmit the reference electromagnetic signal reflective of the test acoustical signal to the implanted hearing aid element;
obtaining feedback from said patient regarding a perception of said transmitted test communication signal; and
adjusting a performance of said hearing aid based on said feedback from said patient.

11. A method as set forth in claim 10, wherein said reference signal output comprises an RF transmitter and said step of operating comprises transmitting an RF signal.

12. A method as set forth in claim 10, wherein said step of adjusting a performance comprises determining internal values for operation of said hearing aid.

13. An apparatus for use in connection with a reference transmitter for testing an implanted element of a hearing aid, said implanted hearing aid element being adapted for directly stimulating a middle ear element of a patient in response to a communication signal transmitted transcutaneously to said implanted hearing aid element, said reference transmitter being operative for receiving an input signal reflecting a test acoustical output of an audiometer, converting said input signal into a test communication signal and transmitting the test communication signal, said apparatus comprising:
an interface for connecting to the reference transmitter to receive the reference transmitter output signal;
a transducer for receiving the reference transmitter output signal and outputting a test signal based on the received reference transmitter output signal, said transducer being adapted for placement over the implanted hearing aid element on a head of a patient; and
a lead for interconnecting the interface and a transducer so as to transmit the reference transmitter output signal from the interface to the transducer.

14. An apparatus as set forth in claim 13, wherein said interface comprises a connector for use in establishing a connection with an output port of said reference transmitter.

15. An apparatus as set forth in claim 13, wherein said transducer comprises an RF transmitter element.

16. An apparatus for use in testing an external portion of a semi-implantable hearing aid, said external portion being adapted for transcutaneously transmitting communication signals to an implanted portion of said hearing aid, said apparatus comprising:
a reference receiver system for receiving an input communication signal from said exterior portion of said hearing aid, wherein said input communication signal is based on a test acoustical signal provided by a hearing aid analyzer;
a signal processor for processing a receiver signal based on said input communication signal to generate an output signal of characteristics corresponding to a microphone signal of an acoustical hearing aid testing system; and
an output port for outputting said output signal to said hearing aid;
wherein said analyzer uses said output signal to evaluate a performance of said exterior portion of said hearing aid.

17. An apparatus as set forth in claim 16, wherein said reference receiver system includes an input port and a reference receiver unit, said port being adapted to support said external portion of said hearing aid at a determined distance from said reference receiver unit and said reference receiver being adapted to receive said input communication signal and provide said receiver signal in response thereto.

18. An apparatus as set forth in claim 16, wherein said receiver signal comprises an electrical signal analogous to the mechanical output of an electromechanical transducer hearing aid element and said signal processor correlates said electrical signal to said output signal.

19. A method for use in testing an exterior portion of a semi-implantable hearing aid, said external portion being adapted for transcutaneously transmitting communication signals to an implanted portion of said hearing aid, said method comprising the steps of:
exposing said exterior portion of said hearing aid to a test acoustical signal generated by a reference acoustic transducer;
interconnecting said exterior hearing aid portion to a reference receiver, said reference receiver being operative to receive an input communication signal from said exterior portion of said hearing aid, process said input communication signal to generate an output signal that corresponds to a microphone signal of an acoustical hearing aid testing system and transmit said output signal to an analyzer that uses the output signal to evaluate a performance of said exterior portion of said hearing aid; and
operating said reference receiver and said analyzer to evaluate a performance of said exterior portion of said hearing aid.

20. A method as set forth in claim 19, wherein said step of exposing comprises disposing said exterior portion of said hearing aid in a test chamber and transmitting said test acoustical signal into said test chamber.

21. A method as set forth in claim 19, wherein said step of interconnecting comprises providing a port for receiving said external portion of said hearing aid, said port having a known geometric relationship to an RF detector of said reference receiver and disposing said external portion of said hearing aid within said port.

22. A method for use in calibrating a hearing aid testing system, said testing system including a reference transmitter and a reference receiver, where the reference transmitter and reference receiver can be used to test the internal and external components, respectively, of a semi-implantable hearing aid, said method comprising the steps of:
interconnecting said reference transmitter to said reference receiver so that the reference receiver receives a transmitter output signal from said reference transmitter;
interconnecting said reference transmitter to an audiometer for receiving an audiometer test signal;
interconnecting said reference receiver to a hearing aid analyzer;
operating said audiometer to generate said audiometer test signal;
operating said reference transmitter to receive said audiometer test signal and generate said transmitter output signal based thereon;
generating said reference receiver to provide a receiver output signal reflective of said transmitter output signal and correlated to a microphone input signal; and
operating said hearing aid analyzer to provide an indication of hearing aid performance, wherein said indication can be used to calibrate said testing system.
